(19) United States
(12) Patent Application Publication
(10) Pub. No.: US 2015/0376566 A1
(43) Pub. Date: Dec. 31, 2015

(54) Process for the Preparation of Multicellular Spheroids

(71) Applicant: SpheroTec GmbH, Martinsried (DE)

(72) Inventor: Barbara Mayer, Memmingen (DE)

(73) Assignee: SpheroTec GmbH, Martinsried (DE)

(21) Appl. No.: 14/846,937

(22) Filed: Sep. 7, 2015

Related U.S. Application Data
(63) Continuation of application No. 12/492,624, filed on Jun. 26, 2009.

(30) Foreign Application Priority Data
Jun. 26, 2008 (EP) 08 011 629.6

(51) Int. Cl. C12N 5/00

(52) U.S. Cl. CPC C12N 5/0062; C12N 2503/02; C12N 2513/00

(57) ABSTRACT

The invention pertains to a process for the preparation of multicellular spheroids from a suspension of single cells, wherein the cells are directly derived from a biological tissue and/or from cell-containing bodily fluid. The invention is further directed to the multicellular spheroids obtained by the process according to the invention as well as to the use of the spheroids for diagnostic, screening and therapeutic purposes.

### CROSS-REFERENCE TO RELATED APPLICATIONS

This application claims Paris Convention Priority of EP 08 011 629.6, filed on Jun. 26, 2008, the disclosure of which is incorporated by reference herein in its entirety and for all purposes.

### FIELD OF THE INVENTION

The invention pertains to a process for the preparation of multicellular spheroids from a suspension of single cells, wherein the cells are directly derived from biological tissues and/or cell-containing bodily fluids. The invention is further directed to the multicellular spheroids obtainable by the process according to the invention as well as to the use of the spheroids for diagnostic, screening and therapeutic purposes.

### BACKGROUND OF THE INVENTION

Tissue culturing has traditionally been used as a model for studying disease processes, e.g., cancer, and also for testing potential therapeutic agents for use in the treatment thereof. Generally, cells used for cell cultures are grown into two-dimensional monolayers on plastic plates covered with a liquid medium which supplies essential nutrients and growth factors for the cells. The cells adhere to the bottom surface of the container, assume a characteristic flattened pattern during spreading, and replicate on that surface as a single layer called a monolayer. The media remains on the top of the flat layer of cells and is changed periodically to provide the growing cells with essential nutrients.

An advantage of this system is that the cells are supplied uniformly with the nutrients. Additionally, while the cells are in culture, various agents can be applied to the media in the plates and the effect on the cells can be observed. For example, suspected carcinogens can be added to individual cultures of cells to ascertain if the carcinogen causes the cells to exhibit a growth pattern characteristic of cancerous cells. The analysis of results can be easily carried out, for example by the use of genetic analysis, chromosome analysis or DNA microarray analysis.

Another possibility is to use a cancer cell line to test the effect of different chemotherapeutics on the cells, thus obtaining information about whether a drug is likely to be useful in a therapeutic regimen for the treatment of cancer.

The two-dimensional cultures are often used for replication of cell lines. When it is desired to split the cultures, an enzyme such as trypsin is utilized to destroy the anchorage of the cells to the dish so that subcultures can be made.

Even though two-dimensional monolayer tissue culture has provided great benefits to scientists and clinicians, it suffers from a lingering disadvantage as well. Cells, such as tumor cells, do not grow two-dimensionally in the body and, therefore, whilst monolayer cultures of cells may reflect the architecture of normal organs, such cultures do not reflect the true in vivo three-dimensional architecture of tumors.

Because all cells in a monolayer system are subjected to the same growth conditions, this leads to some disadvantages. Namely the resulting culture represents a homogenous cell population wherein every cell is substantially similar to every other cell in the culture. In contrast, naturally occurring cells generally represent a heterogeneous cell population resulting, for example, from positional cues, cell differentiation induced by differences in cellular interactions and the biochemical environment such as hormones, growth factors, oxygen tension, etc.

In an attempt to mimic the conditions in which cells develop in vivo, three-dimensional cell culture systems have been developed and used for decades in medical and biologic research. In 1944, first experiments with respect to morphogenesis of amphibian embryos were performed in a three-dimensional cell culture (J. Holtfreter, A study of the mechanics of gastrulation, J. Exp. Zool., 1944, 95: 171-212). Also, embryonic cells (A. Moscana, Cell suspensions from organ rudiments of chick embryos, Exp. Cell Res., 1952, 3: 535-539) and ex vivo tumor cells (A. Moscana, The development in vitro of chimeric aggregates of dissociated embryonic chick and mouse cells, Proc. Natl. Acad. Sci. (USA), 1957, 43: 184-104) have been used.

Nowadays, it is preferable to use well-established cell lines since this allows standardization and thus comparability of the results between experiments and laboratories.

To prepare and cultivate cell cultures that mimic the in vivo three-dimensional tissue architecture, a number of methods have been developed. These include the spinner flask technique, the liquid-overlay technique, the high aspect rotating vessel technique and the hanging drop method to name but a few.

Generally, these methods involve cultivation of adherent growing cells in vessels with a non-adherent surface, wherein cell aggregation is either induced through movement or wherein single cells are grown into colonies in soft agar or utilizing collagen, fibronectin, laminin or other molecules derived from the extracellular cell matrix (ECM).

WO 95/34637 discloses a method for inducing expression of biomarkers for urologic cancers, including prostate and bladder cancer. Tumor cells are cultured using a three-dimensional technique under conditions effective to induce said expression. The method can be used for diagnostic and therapeutic applications.

A three-dimensional cell culture preparation method is also disclosed in WO 2004/101743 A2 and WO 2005/095585 A1.

Although the three-dimensional cell culture systems of the prior art represent a valuable transition from the two-dimensional cell culture towards mimicking the in vivo cell system, the three-dimensional systems of the prior art continue to suffer from a number of disadvantages with the result that they are still too far removed from the in vivo system.

Thus, there is a need for cell culture systems and methods that more effectively mimic the three dimensional cellular relationships and environment of cells in vivo.

### SUMMARY OF THE INVENTION

The present invention provides a process for the preparation of multicellular spheroids comprising
a) preparing a suspension of single cells from a biological tissue and/or cell containing bodily fluid in a medium,
b) adjusting the concentration of cells in the single cell suspension within the range of from 10³ to 10⁷ cells/ml medium,
c) adding 2 to 50 vol.-% of an inert matrix to the suspension of single cells,
d) incubating the suspension of single cells,
   characterized in that the suspension of single cells is directly derived from at least one biological tissue and/or from at least one cell-containing bodily fluid.

Surprisingly it has been found that multicellular spheroids obtained according to the process of the present invention, exhibit an antigen and genetic profile which substantially mimics that of the cells of the biological tissue and/or cell-containing bodily fluid of origin.

Thus, the multicellular spheroids according to the present invention are an ideal system for studying e.g., the effect(s) of chemical compounds such as drugs on the cells. The multicellular spheroids according to the present invention are particularly useful, for example, in diagnosis or determination/suggestion of therapeutic strategies, pharmacokinetic profiling, pharmacodynamic profiling, identification and circumvention of therapeutic resistance, investigations into treatment strategies such as chemotherapy, radiotherapy, hyperthermia or molecular targeted therapies, biomarker identification, tumor profiling, personalised or tailored therapies, testing and/or identification of small molecules, therapeutic proteins and scaffolds, RNA and DNA ‘drugs’, drug penetration studies and antibody generation.

### DETAILED DESCRIPTION OF THE INVENTION

Throughout this application, various articles and patents are referenced. Disclosures of these applications in their entirety are hereby incorporated by reference into this application.

As used herein, the term three-dimensional cell culture refers to any method usable to effect the growth of cells in a three-dimensional multicellular form such as spheroids.

As used herein, the term “spheroid” refers to an aggregate, cluster or assembly of cells cultured to allow three-dimensional growth in contrast to the two-dimensional growth of cells in either a monolayer or cell suspension (cultured under conditions wherein the potential for cells to aggregate is limited). The aggregate may be highly organized with a well defined morphology or it may be a mass of cells that have clustered or adhered together with little organisation reflecting the tissue of origin. It may comprise a single cell type (homotypic) or more than one cell type (heterotypic). Preferably the cells are primary isolates but may also include a combination of primary isolates with an established cell line(s). Particular cell ‘types’ include somatic cells, stem cells, progenitor cells and cancer stem cells.

As used herein, the term “directly derived” refers to a suspension of single cells from a biological tissue and/or cell containing bodily fluid that has been obtained directly from an individual, donor patient or animal without intermediate steps of subculture through a series of cultures and/or hosts. Thus, a suspension of single cells is produced directly from the biological tissue and/or cell-containing bodily fluid. This is in contrast to established methods in which stable and highly passaged cell lines are used. Such cell lines are far removed from being directly derived from their progenitor tissue by several, often a great many, intermediate culture steps. By way of non-limiting example, sources of suitable tissues include benign or malignant primary and metastatic tissues, sources of suitable cell containing bodily fluids include pleural effusion fluid or ascites fluid (liquid tumors).

A “primary culture” is an initial culture of cells freshly isolated from a tissue.

The term “cell line” as used herein refers to cells derived from a primary culture by subculturing and that have exceeded the Hayflick limit. The Hayflick limit may be defined as the number of cell divisions that occur before a cell line becomes senescent or unable to replicate further. This limit is approximately 50 divisions for most non-immortalized cells and in terms of cell culture, equates to approximately 9 to 10 passages of cell subculture over the course of from about 12 to 14 weeks.

Primary tumors are tumors from the original site where they first developed. For example, a primary brain tumor is one that arose in the brain. This is in contrast to a metastatic tumor that arises elsewhere and metastasized or spread to, for example, the brain.

According to the invention the tissue which may be used for spheroid preparation may be a normal or healthy biological tissue, or may be a biological tissue afflicted with a disease or illness, such as a tissue or fluid derived from a tumor. Preferably the tissue is a mammalian tissue. Also encompassed are metastatic cells. The tissue may be obtained from a human, for example from a patient during a clinical surgery or from biopsies. The tissue may also be obtained from animals such as mice, rats, rabbits, and the like. It is also possible according to the invention to prepare spheroids from stem cells, progenitor cells or cancer stem cells.

Besides cells originating from tumor tissue, other cells with various indications such as smooth muscle cells, adipocytes, neural cells, stem cells, islet cells, foam cells, fibroblasts, hepatocytes and bone marrow cells, cardiomyocytes and enterocytes are also encompassed within the present invention.

Also within the scope of the present invention is the possibility to rebuild a metastatic microtumor e.g., tumor cells with hepatocytes, or tumor cells with bone marrow cells.

Also useful within the invention are primary cancer cells such as gastric, colon and breast primary cancer cells and metastatic cells. Also encompassed by the invention are primary normal (healthy) cells such as endothelial cells, fibroblasts, liver cells, and bone marrow cells.

Preferably the cells are directly derived from the tissue of a patient or healthy donor, a tissue derived from a biopsy, surgical specimens, an aspiration or a drainage and also cells from cell-containing bodily fluids.

The prior art methods disclose preparation of spheroids from well known cell lines which may be proliferated multiple times. As a result, the cell lines used in the prior art represent homogeneous cell lines which are not able to mimic a more heterogeneous in vivo cell system. Thus, the process of the present invention represents a significant step forward over the prior art since it has previously been extremely difficult to produce spheroids reliably and in useful quantities from “directly derived” or primary isolate samples.

Also within the scope of the invention are large spheroids which consist of a higher cell number in the range of from 10⁶ to 5×10⁶ cells. Large spheroids generally have a necrotic/apoptotic centre that correlates with the upregulation of various biomarkers such as HIF-1alpha, VEGF, TKTL-1 and others. Large and small spheroids are generally used for different purposes, for example, large spheroids may be used as a model of advanced tumors.

The present invention comprises a process for the preparation of multicellular spheroids. In a first step of the method a suspension of single cells is prepared from at least one biological tissue and/or cell-containing bodily fluid in a medium. The concentration of cells in the suspension is adjusted in the range of from 10³ to 10⁷ cells/ml medium. 2 to 50 vol.-% of an inert matrix is then added to the suspension of single cells, which is then incubated, preferably in the presence of CO₂. The process is characterized in that the suspension of single cells is directly derived from at least one biological tissue and/or from at least one cell-containing bodily fluid.

In the process according to the invention the cells of the biological tissue and/or cell containing bodily fluid are first dissociated or separated from each other. Dissociation of the tissue is accomplished by any conventional means known to those skilled in the art. Preferably the tissue is treated mechanically or chemically, such as by treatment with enzymes. More preferably the tissue is treated both mechanically and enzymatically. Use of the term ‘mechanically’ means that the tissue is treated to disrupt the connections between associated cells, for example, using a scalpel or scissors or by using a machine, such as a homogenizer. Use of the term ‘enzymatically’ means that the tissue is treated using one or more enzymes to disrupt the connections between associated cells, for example, by using one or more enzymes such as collagenase, dispases, DNAse and/or hyaluronidase. Preferably a cocktail of enzymes is used under different reaction conditions, such as by incubation at 37° C. in a water bath or at room temperature with shaking.

The dissociated tissue is then suspended in a medium to produce a suspension of single cells and from which the spheroids can be formed directly. It should be noted that prior art methods generally include a step of two-dimensional tissue culture in a growth medium prior to attempting three-dimensional cell cultivation. In two-dimensional culturing methods the cell culture adheres to the bottom of a vessel and is then removed for example with Trypsin, EDTA, Bionase or other suitable agents. After this 2D culture step, the suspension of single cells is prepared and from which the spheroids are produced. Thus, the two-dimensional culturing according to the prior art leads to a more or less homogenous cell culture which is accordingly not able to mimic a heterogeneous in vivo cell system.

In contrast thereto, it has surprisingly been found that spheroids produced from suspensions of single cells prepared from primary isolate tissue according to the present invention retain essentially all of the biological properties of the originating biological tissue. This is the case for both homotypic and heterotypic cell systems. The same applies when cell-containing bodily fluids are used.

Preferably the suspension of single cells is treated to remove dead and/or dying cells and/or cell debris. The removal of such dead and/or dying cells is accomplished by any conventional means known to those skilled in the art for example, using beads and/or antibody methods. It is known, for example, that phosphatidylserine is redistributed from the inner to the outer plasma membrane leaflet in apoptotic or dead cells. Annexin V and any of its conjugates which have a high affinity for phosphatidylserine can therefore be bound to these apoptotic or dead cells. The use of Annexin V-Biotin binding followed by binding of the biotin to streptavidin magnetic beads enables separation of apoptotic cells from living cells. Other suitable methods will be apparent to the skilled artisan. Surprisingly it has been found that as a result of the inclusion of this step, substantially all of the cells within the suspension of single cells are available to form spheroids with a biological profile or composition more closely mimicking that found in vivo.

Methods of the prior art often utilize a dye exclusion test to monitor the vitality or viability of cells. The dye exclusion test is used to determine the number of viable cells present in a cell suspension. It is based on the principle that live cells possess intact cell membranes that exclude certain dyes, such as trypan blue, eosin, or propidium iodide, whereas dead cells do not. In the trypan blue test, a cell suspension is simply mixed with dye and then visually examined to determine whether cells take up or exclude dye. A viable cell will have a clear cytoplasm whereas a nonviable cell will have a blue cytoplasm. Dye exclusion is a simple and rapid technique measuring cell viability but it is subject to the problem that viability is being determined indirectly from cell membrane integrity. Thus, it is possible that a cell's viability may have been compromised (as measured by capacity to grow or function) even though its membrane integrity is (at least transiently) maintained. Conversely, cell membrane integrity may be abnormal yet the cell may be able to repair itself and become fully viable. Another potential problem is that because dye uptake is assessed subjectively, small amounts of dye uptake indicative of cell injury may go unnoticed. In this regard, dye exclusion performed with a fluorescent dye using a fluorescence microscope may result in the scoring of more nonviable cells with dye uptake than tests performed with trypan blue using a transmission microscope. As a result of the use of this method, the suspensions of single cells and spheroids of the prior art comprise a far greater proportion of apoptotic or dead cells. This inclusion of dead matter means that the prior art spheroids are less able to mimic the conditions found in biological tissue in vivo.

A more sophisticated method of measuring cell viability is to determine the cell's light scatter characteristics, 7AAD or propidium iodide uptake. It will be apparent to one skilled in the art that use of a flow cytometer coupled with cell sorting may also accomplish removal of dead and/or apoptotic cells.

The suspension of single cells is prepared in a culture medium. The medium is designed such that it is able to provide those components that are necessary for the cell's survival. Preferably the suspension of single cells is prepared in a medium comprising one or more of the following components: serum, buffer, interleukins, chemokines, growth factors, hydrogen carbonate, glucose, physiological salts, amino acids and hormones.

A preferred medium is RPMI 1640. RPMI 1640 was developed by Moore et. al. at Roswell Park Memorial Institute (hence the acronym RPMI). The formulation is based on the RPMI-1630 series of media utilizing a bicarbonate buffering system and alterations in the amounts of amino acids and vitamins. RPMI 1640 medium has been used for the culture of human normal and neoplastic leukocytes. RPMI 1640, when properly supplemented, has demonstrated wide applicability for supporting growth of many types of cultured cells.

Preferably, the medium further comprises L-glutamine, in particular a stabilized L-glutamine. L-glutamine is an essential nutrient in cell cultures for energy production as well as protein and nucleic acid synthesis. However, L-glutamine in cell culture media may spontaneously degrade, forming ammonia as a by-product. Ammonia is toxic to cells and can affect protein glycosylation and cell viability, lowering protein production and changing glycosylation patterns. It is thus preferred that the L-glutamine is a stabilized glutamine, most preferably it is the dipeptide L-alanyl-L-glutamine, which prevents degradation and ammonia build-up even during long-term cultures. The dipeptide is commercially available as Glutamax I™ (Invitrogen, Carlsbad, Calif.).

The medium may further comprise additional components such as antibiotics, for example, penicillin, streptomycin, neomycin, ampicillin, metronidazole, ciprofloxacin, gentamicin, Amphotericin B, Kanamycin, Nystatin; amino acids such as methionine or thymidine; FCS and the like.

In addition to, or instead of, RPMI1640 other liquid media can be used, for example DMEM high or low glucose, Ham's F-10, McCOY's 5A, F-15, RPMI high or low glucose, Medium 199 with Earle's Salts or the different variants of MEM Medium.

In a next step of the method, the concentration of cells in the suspension is adjusted to an appropriate cell concentration. An appropriate cell concentration means an amount of cells per millilitre of culture medium which supports the formation of spheroids in the incubation step. Appropriate cell amounts are preferably 10³ to 10⁷ cells/ml medium, more preferably 10³ to 5×10⁶ cells/ml medium and most preferred 10⁵ to 10⁶ cells/ml medium. Methods of determining cell concentration are known in the art, for example, the cells may be counted with a Neubauer counter chamber (hemocytometer).

In a next step of the process of the present invention an appropriate amount of an inert matrix is added to the suspension of single cells. Use of the term “inert” as used herein refers to a matrix that has limited or no ability to react chemically and/or biologically, i.e., having little or no effect on the biological behaviour or activity of the cells in the suspension. Ideally the inert matrix is of non-human origin.

Preferably the inert matrix increases the viscosity of the culture medium. Not wishing to be bound by theory, it is believed that increasing the viscosity of the culture fluid increases the co-incidental collision and adherence of cells with each other resulting in the formation of aggregates. This is particularly useful since it improves the ability of shear sensitive or weakly adherent cells to aggregate and develop into spheroids.

Thus, the inert matrix supports or promotes the formation of spheroids during the incubation step. Preferably the inert matrix is added to the culture medium in an amount of 2 to 50 vol.-% based on the total volume of the medium. Preferably the inert matrix is added in an amount of 5 to 30 vol.-%, most preferably in an amount of 10 to 15 vol. %. Particular amounts will vary depending on the source or composition of the cells such as 3, 4 or 5 vol. % up to 10 or 15 vol.-% for cell lines and up to 30 to 45 or 50 vol. % when using primary isolate tissue. These amounts are based on the total volume of the medium. The inert matrix is preferably a non-ionic poly(ethylene oxide) polymer, water soluble resin or water soluble polymer such as a cellulose ether. Preferably the inert matrix is selected from the group comprising carboxymethyl cellulose, hydroxypropyl cellulose, hydroxypropylmethyl cellulose, hypomellose, methyl cellulose, methylethyl cellulose. However, also suitable is cellulose, agarose, seaplaque agarose, starch, tragacanth, guar gum, xanthan gum, polyethylene glycol, and the like.

In the next process step of the present invention the single cell suspension is incubated, preferably in the presence of CO₂. Incubation can also be carried out in the presence of water vapour. Possible preparation techniques are e.g., the liquid-overlay technique, the spinner flask technique, the high aspect rotating vessel (HARV) technique or the hanging drop method. These methods are known to the skilled artisan. The HARV technique is inter alia disclosed in U.S. Pat. Nos. 5,153,131, 5,153,132, 5,153,133, 5,155,034, and 5,155,035. The spinner flask technique is disclosed in e.g., W. Mueller-Klieser, “Multicellular Spheroids”, J. Cancer Res. Clin. Oncol., 12: 101-122, 1986. The liquid-overlay technique is disclosed e.g., in J. M. Yuhas et. al., “A simplified method for production and growth of multicellular tumor spheroids”, Cancer. Res. 37: 3639-3643, 1977. The hanging drop method is disclosed in e.g., Bulletin of Experimental Biology and Medicine, Vol. 91, 3, 1981, Springer, New York. Most preferred in the present invention is the liquid-overlay technique. Generally these preparation techniques are all performed under CO₂ conditions.

The incubation may be performed at 30 to 45° C., preferably at 37° C., in a normoxic atmosphere containing about 4 to 6 vol.-% CO₂, preferably 5 vol. % CO₂ or under hypoxic conditions, i.e., N₂ 92-95%, O₂ 5-8% . The incubation is performed from about 5 hours to 9 days, preferably of from about 12 hours to 6 days, most preferred of from about 24 to about 96 hours. However, it will be apparent to the skilled artisan that such temperatures and conditions will depend on the source and type of cells used.

As used herein, the term ‘homotypic’ refers to cells of a single type. For example, commercially available cell lines are generally homotypic. In contrast and as used herein, the term ‘heterotypic’ refers to cells of more than one cell type. For example, primary isolate tissue comprising different cell types will be heterotypic.

Thus, a homotypic spheroid could be prepared from one particular cancer cell line such as Hs746T, malignant primary cells isolated from primary or metastatic malignant tissues or healthy, benign primary cells such as fibroblasts, bone marrow cells, hepatocytes, other benign epithelial cells or chondrocytes. Heterotypic spheroids could be prepared from primary and metastatic patient tissue or from a combination of two or more homotypic cell lines such as a cancer cell line and benign primary cells.

Therefore, in another aspect of the present invention, a suspension of single homotypic cells may be combined with, for example, epithelial cells, immune cells, fibroblasts, hepatocytes, chondrocytes, bone marrow cells, airway epithelial cell cultures, enterocytes, cardiomyocytes, melanocytes, keratinocytes, adipocytes, stem cells, cancer stem cells and/or smooth muscle cells, resulting in the formation of heterotypic spheroids. It will be apparent to one skilled in the art that a suspension of single heterotypic cells may also be combined in this manner.

The advantage of combining homotypic cell types with other cell types, such as epithelial cells, immune cells, fibroblasts (as well as other cell types cited above), is that tumour cells interact with epithelial cells, immune cells and/or fibroblasts (and also with such cells cited above) in nature. Hence, the combination with such cells leads to a heterotypic, multicellular spheroid system which mimics even more closely an in vivo cell or metastic cell system.

The internal environment of a spheroid is dictated by the metabolism and adaptive responses of cells with a well-defined morphological and physiological geometry. Most homotypic spheroids develop concentric layers of heterogeneous cell populations with cells at the periphery and layers of quiescent cells close to a necrotic core. The heterogeneous arrangement of cells in a spheroid mimics initial avascular stages of early tumours. Although homotypic spheroids are able to mimic closely the in vivo morphology, some of the biological complexity is lost. Thus, by co-culturing more than one cell type, tumour cell interactions with other cell types reflecting natural cell interaction in vivo can be established better representing the in vivo environment.

Thus, suspensions of single cells may be combined with other cells, for example from established cell lines, primary cells and/or primary or metastatic tissues. Most preferably the tissue is a tumour tissue wherein the cancer cell lines may be cell lines from gastric (e.g., Hs-746T, MKN-28, N87, and the like), colorectal (e.g., HT-29, HCT-116, DLD-1, and the like), liver (e.g., HepG2, and the like), pancreas (e.g., L.6p1, AsPC-1, MiaPACA, and the like), lung (e.g., A549, H358, H1299, and the like), kidney (e.g., 786-O, A-498, CAKI-1, and the like), breast (e.g., MCF-7, BT549, Hs575T, and the like), cervical (e.g., HeLa, and the like), prostate (e.g., PC-3, LNCaP, DU-145, and the like) or glioma (e.g., U251, U373, and the like) cell lines. It will be appreciated that the method is suitable for use with any cell line. In particular preferred are also cell lines from sarcoma or astrocytoma tissue.

Another aspect of the invention is a multicellular spheroid, which is obtained by the process according to the invention. The spheroid may comprise a single cell type (homotypic) or a mixture of two or more cell types (heterotypic).

The process as set forth above leads to spheroids with a nearly homogenous spherical shape, wherein the average diameter of the spheroids reaches from 50 to 2000 μm, preferably from 150 to 1000 μm and most preferred from 200 to 500 μm.

The multicellular spheroids according to the invention can also be characterised in that they exhibit characteristics that substantially mimic those of the tissue of origin, such as: antigen profile and/or genetic profile, tumour biologic characteristics, tumour architecture, cell proliferation rate(s), tumour microenvironments, therapeutic resistance and composition of cell types. Preferably, they exhibit an antigen profile and genetic profile which is substantially identical to that of the tissue of origin.

Thus, the spheroids of the invention exhibit a substantially similar/identical behaviour to that of natural cell systems, e.g., with respect to organization, growth, viability, cell survival, cell death, metabolic and mitochondrial status, oxidative stress and radiation response as well as drug response.

Since the multicellular spheroids according to the invention are substantially identical to in vivo cell systems, these spheroids can thus be used for diagnostic and/or therapeutic purposes, for example, pharmacokinetic profiling, pharmacodynamic profiling, efficacy studies, cytotoxicity studies, penetration studies of compounds, therapeutic resistance studies, antibody generation, personalized or tailored therapies, RNA/DNA ‘drug’ testing, small molecule identification and/or testing, biomarker identification, tumour profiling, hyperthermia studies, radioresistance studies and the like.

In one aspect, the multicellular spheroids can be obtained from benign or malignant tissues or from primary cells and used for the screening of compounds, for example, as new therapeutic agents or screening for e.g., chemotherapeutica wherein the response of the spheroid to the chemotherapeuticum can be determined. It is thus possible to see whether a chemotherapeuticum has an effect and/or side effects on the multicellular spheroid, e.g., whether it causes cell death (apoptosis) or other biologic effect.

In the sense of the present invention, preferably the term “chemotherapeutica” should be understood as to include all chemical substances used to treat disease. More particularly, it refers to antineoplastic drugs used to treat cancer or the combination of these drugs into standardized treatment regimen. In its non-oncological use, the term may also refer to antibiotics (antibacterial chemotherapy). Other uses of cytostatic chemotherapy agents are the treatment of autoimmune diseases such as multiple sclerosis and rheumatoid arthritis, viral infections, heart diseases and the suppression of transplant rejections. It will of course be apparent to the skilled artisan that such chemotherapeutica need not be limited to substances used to treat disease. Thus, the term may be applied more loosely to refer to any agent that the skilled person wishes to expose the spheroids to determine whether said agent has an effect, for example, on the behaviour or biological characteristics of the spheroids.

By way of non-limiting example, chemotherapeutic agents may include: alkylating agents, antimetabolites, anthracyclines, plant alkaloids, topoisomerase inhibitors, and other anti-tumour agents, antibodies such as monoclonal, single chain or fragments thereof and the new tyrosine kinase inhibitors e.g., imatinib mesylate (Gleevec® or Glivec®) (Novartis AG, Basel, Switzerland) small molecules, tyrosine kinase receptor inhibitors, anticalins, aptamers, peptides, scaffolds, biosimilars, generic drugs, siRNA and RNA or DNA based agents.

The present invention will now be more fully described by way of examples that are intended to aid understanding of the invention, but are not intended, and should not be construed, to limit the scope of the invention in any manner.

### EXAMPLES

### Example 1

### Preparation of Heterotypic Spheroids Derived from Primary Patient Tumour Tissue

A gastric cancer tissue with a size of about 0.5 cm³ was taken from a patient. The tissue was made into a suspension of single cells by reducing the tissue into small pieces with the aid of a scalpel and subsequent treatment with an enzyme cocktail consisting of collagenase, dispase, DNAse and hyaluronidase. Then, the cells were suspended in RPMI 1640 culture medium containing Glutamax I™ (Invitrogen, Carlsbad, Cailf.) or L-Glutamine.

The viability of the cells was tested with the trypan-blue exclusion test and the cell number was adjusted to 10⁶ cells/ml medium with the aid of a Neubauer counter chamber. Cellulose ether was then added to the cell suspension and the suspension transferred to a 96-well plate with the following amounts of reagents:

For each well plate 12 ml cell suspensions were prepared (96 well×100 μl/well=−10 ml+2 ml excess=12 ml), to provide a concentration of cells of 5×10⁴ cells/100 μl medium corresponding to 5×10⁵/ml corresponding to 6×10⁶/12 ml.

The final suspension contained 6 ml of cell suspension, 5.5 ml RMPI 1640+Glutamax™ and 0.6 ml cellulose ether (=5%)

The cell suspension was mixed and then transferred with a multichannel pipette to a 96-well plate in an amount of 100 μl/well. The cell suspension was then incubated at 37° C. in the presence of 5% CO₂ for 24 hours.

After 24 hours, multicellular spheroids had formed and exhibited a homogeneous shape with a mean diameter of about 250 μm.

### Example 2

### Preparation of Heterotypic Spheroids by Combining a Homotypic Cell Line With a Primary Cell Type

Homotypic cells from a human gastric carcinoma cell line (Hs746T) were suspended in RPMI 1640 culture medium containing Glutamax I™ or L-Glutamine.

The viability of the cells was tested with the trypan-blue exclusion test and the concentration of cells was adjusted to 10⁶ cells/ml medium with the aid of a Neubauer counter chamber. Cellulose ether was then added to the cell suspension and the suspension transferred to a 96-well plate with the following amounts of reagents:

For each well plate 12 ml cell suspension was prepared (96 well×100 μl/well=−10 ml+2 ml excess=12 ml), to provide a concentration of cells of 5×10⁴ cells/100 μl medium corresponding to 5×10⁵/ml corresponding to ×10⁶/12 ml.

The final suspension contained 6 ml of the cell suspension, 5.5 ml RMPI 1640+Glutamax™ and 0.6 ml cellulose ether (=5%).

The Hs746T cell suspension was mixed with fibroblasts (in either a 1:1 ratio or 9:1 ratio) and transferred with a multichannel pipette to a 96-well plate in an amount of 100 μl/well. The cell suspension was then put in an incubator and incubated at 37° C. in the presence of 5% CO₂ for 24 hours.

After 24 hours heterotypic multicellular spheroids had formed. The spheroids comprised both cells of the Hs746T cell line and fibroblast cells and exhibited a homogeneous shape with a mean diameter of about 200 μm.

### Example 3

### Preparation of Heterotypic Spheroids by combining a Homotypic Cell Line With Two or More Different Primary Cell Types, e.g., the Gastric Cancer Cell Line Hs746T Cocultured With Immune Cells and Hepatocytes

Homotypic cells from a human gastric carcinoma cell line (Hs746T) were suspended in RPMI 1640 culture medium containing Glutamax I™ or L-Glutamine.

The viability of the cells was tested with the trypan-blue exclusion test and the concentration of cells was adjusted to 10⁶ cells/ml medium with the aid of a Neubauer counter chamber. Cellulose ether was then added to the cell suspension and the suspension transferred to a 96-well plate with the following amounts of reagents:

For each well plate 12 ml cell suspension was prepared (96 well×100 μl/well=−10 ml+2 ml excess=12 ml), to provide a concentration of cells of 5×10⁴ cells/100 μl medium corresponding to 5×10⁵/ml corresponding to 6×10⁶/12 ml.

The final suspension contained 6 ml of the cell suspension, 5.5 ml RMPI 1640+Glutamax™ and 0.6 ml cellulose ether (=5%).

The Hs746T cell suspension was mixed with two cell types, immune cells and hepatocytes, derived from primary tissues (in either a 1:1 ratio or 9:1 ratio) and transferred with a multichannel pipette to a 96-well plate in an amount of 100 μl/well. The cell suspension was then put in an incubator and incubated at 37° C. in the presence of 5% CO₂ for 24 hours.

After 24 hours heterotypic multicellular spheroids had formed. The spheroids comprised both cells of the Hs746T cell line and both immune cells and hepatocytes. The heterotypic spheroids exhibited a homogeneous shape with a mean diameter of about 200 μm.

What is claimed:

1. A process for the preparation of multicellular spheroids, comprising
a) preparing in a medium, a suspension of single cells from at least one biological tissue or cell-containing bodily fluid,
b) adjusting the concentration of cells in the suspension to a concentration in the range of from 10³ cells to 10⁷ cells,
c) adding 2 to 50 vol.-% of an inert matrix to the suspension of single cells, and
d) incubating the suspension of single cells,
wherein the suspension of single cells is directly derived from at least one biological tissue or cell-containing bodily fluid.

2. The process according to claim 1, wherein the biological tissue is a healthy tissue, a tumour tissue, a benign primary tissue, or a malignant primary tissue.

3. The process according to claim 1, further comprising treating the suspension of single cells to remove dead and/or dying cells and/or cell debris.

4. The process according to claim 1, wherein the biological tissue is a mammalian tissue.

5. The process according to claim 1, wherein the biological tissue is treated mechanically and/or enzymatically before preparing the suspension of single cells.

6. The process according to claim 1, wherein the single cell suspension is prepared in a medium comprising serum, buffer, interleukins, chemokines, growth factors, hydrogen carbonate, glucose, physiological salts, amino acids and hormones.

7. The process according to claim 6, wherein the medium further comprises at least one antibiotic selected from the group consisting of penicillin, streptomycin, neomycin, ampicillin, metronidazole, ciprofloxacin, gentamicin, Amphotericin B, Kanamycin and Nystatin.

8. The process according to claim 1, wherein the concentration of single cells is adjusted to 10³ to 10⁷ cells/ml medium.

9. The process according to claim 1, wherein the inert matrix is added in an amount of from 2 to 50 vol.-% based on the total volume of the medium.

10. The process according to claim 1, wherein the inert matrix is derived from a non-human source.

11. The process according to claim 1, wherein the inert matrix is selected from the group comprising cellulose ether, carboxymethyl cellulose, hydroxypropyl cellulose, hydroxypropylmethyl cellulose, hypomellose, methyl cellulose, methylethyl cellulose, polyethylene glycol, and agarose.

12. The process according to claim 1, wherein the incubation is performed at about 37° C. in an atmosphere containing from 4 to 6 vol.-% CO₂.

13. The process according to claim 1, wherein the incubation is performed from about 12 hours up to about 9 days.

14. The process according to claim 1, wherein the suspension of single cells is combined with at least one cell type selected from the group comprising of epithelial cells, immune cells, hepatocytes, chondrocytes, bone marrow cells, airway epithelial cell culture, melanocytes, keratinocytes, adipocytes, smooth muscle cells, fibroblasts, enterocytes, stem cells, cancer stem cells and cardiomyocytes.

15. A multicellular spheroid, obtained by the process of claim 1.

16. The multicellular spheroid according to claim 15, wherein the at least one biological tissue is generated from benign or malignant primary or metastatic tissue.

17. A multicellular spheroid, comprising a homogeneous spherical shape with an average diameter of from 50 to 2000 μm.

18. The multicellular spheroid according to claim 15, wherein at least one of the antigen profile, genetic profile, tumor biologic characteristics, tumor architecture, cell proliferation rate(s), tumor microenvironments, therapeutic resistance or composition of the cell is substantially identical to that of the tissue of origin.

19. The multicellular spheroid according to claim 15, wherein the antigen profile and the genetic profile of the spheroid is substantially identical to the antigen profile and the genetic profile of the tissue of origin.

20. A method for determining the biological effect of a chemical compound on a cell, comprising contacting the multicellular spheroid of claim 15 with a chemical compound, and determining a biological effect on the multicellular spheroid compared with a multicellular spheroid that has not been contacted with the chemical compound.
